(19) United States
(12) Patent Application Publication
(10) Pub. No.: US 2008/0249132 A1
(43) Pub. Date: Oct. 9, 2008

(54) Piperidine Carboxyamide Derivate Suitable as Tachykinin Receptor Antagonist

(76) Inventor: Silvia Martinucci, Verona (IT)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/995,880

(22) PCT Filed: Jul. 14, 2006
(86) PCT No.: PCT/EP2006/007117

§ 371 Date: Jan. 16, 2008

(30) Foreign Application Priority Data
Jul. 18, 2005 (GB) 0514707.9

(51) Int. Cl. A61K 31/4468; C07D 211/58; A61P 25/00; A61P 29/00

(52) U.S. Cl. 514/329; 546/224

(57) ABSTRACT

The present invention relates to the compound of formula (I)

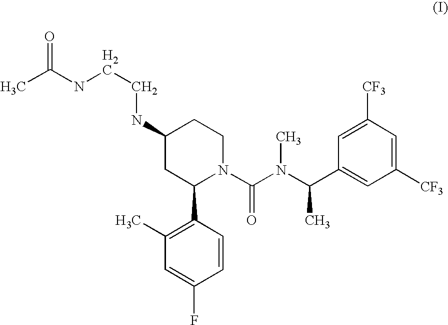

or pharmaceutically acceptable salts or solvates thereof, process for their preparation, their use in the treatment of conditions mediated by tachykinins and pharmaceutical compositions containing them.

The present invention relates to a [(4-acetylamino)ethyl] amino 1-piperdinyl derivative, salts and solvates thereof, to processes for their preparation, to pharmaceutical compositions containing them and to their medical use.

WO 03/099787 discloses some piperidine derivatives antagonists of tachykinins, including substance P and other neurokinins.

Thus, the present invention provides, in one aspect, the compound of formula (I), (2R,4S)-4-{[2-(acetylamino)ethyl]amino}-N-{( 1 R)-1 -[3,5-bis(trifluoromethyl)phenyl]ethyl}-2-(4-fluoro-2-methylphenyl)-N-methyl-1 -piperidinecarboxamide

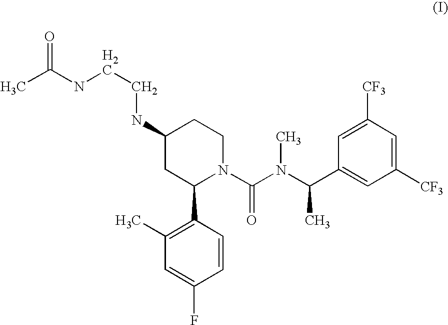

or a pharmaceutically acceptable salt or solvate thereof.

Suitable pharmaceutically acceptable salts of the compound of general formula (I) include acid addition salts formed with pharmaceutically acceptable organic or inorganic acids, for example hydrochlorides, hydrobromides, sulphates, alkyl- or arylsulphonates (e.g. methansulphonates or p-toluenesulphonates), phosphates, trifluoroacetates, acetates, citrates, succinates, tartrates, lactates, malates, fumarates and maleates.

The solvates may, for example, be hydrates.

For the avoidance of doubt, an isolated form of the compound of formula (I) or a pharmaceutically acceptable salt or solvate thereof does not include the compound of formula (I) produced in vivo by the metabolism of 4-(S)-(4-Acetyl-piperazin-1-yl)-2-(R)-(4-fluoro-2-methyl-phenyl)-piperidine-1-carboxylic acid, [1-(R)-(3,5-bis-trifluoromethyl-phenyl)-ethyl]-methylamide.

In another aspect, the invention provides a substantially pure compound of formula (I) or a pharmaceutically acceptable salt or solvate thereof.

In the compound of formula (I), the wedge shaped bond indicates that the bond is above the plane of the paper and is referred to as β configuration.

The β configuration at the 2 position corresponds to the R configuration and the β configuration at the 4 position corresponds to the S configuration. The assignment of the R or S configuration at the 2 and the 4 positions has been made according to the rules of Cahn, Ingold and Prelog, Experientia 1956, 12, 81.

Furthermore, the compound of formula (I) may exist in one or more crystalline forms and the crystalline forms of the compound of formula (I) may exist as polymorphs, which are included in the present invention.

The compound of the invention is a human metabolite of 4-(S)-(4-Acetyl-piperazin-1-yl)-2-(R)-(4-fluoro-2-methyl-phenyl)-piperidine-1-carboxylic acid, [1-(R)-(3,5-bis-trifluoromethyl-phenyl)-ethyl]-methylamide which is described in WO 02/32867.

The compound of the invention is itself an antagonist of tachykinin receptors, including substance P and other neurokinins, both in vitro and in vivo and is thus of use in the treatment of conditions mediated by tachykinins, including substance P and other neurokinins.

Tachykinins are a family of peptides that share a common carboxyl-terminal sequence (Phe-X-Gly-Leu-Met-NH2). They are actively involved in the physiology of both lower and advanced lifeforms. In mammalian lifeforms the main tachykinins are substance P (SP), Neurokinin A (NKA) and Neurokinin B (NKB) which act as neurotransmitters and neuromodulators. Mammalian tachykinins may contribute to the pathophysiology of a number of human diseases.

Three types of tachykinins receptors have been identified, namely NK1(SP-preferring), NK2 (NKA-preferring) and NK3 (NKB-preferring) which are widely distributed throughout the central nervous (CNS) and peripheral nervous system.

Particularly, the compound of the invention is an antagonist of the NK1 receptor.

NK₁-receptor binding affinity has been determined in vitro in filtration binding assay by measuring the compounds' ability to displace [³H]-Substance P (SP) from recombinant human NK₁ receptors stably expressed in Chinese Hamster Ovary (CHO) cell membranes prepared by using a modification of the method described by Beattie D. T. et al. (Br. J. Pharmacol, 116:3149-3157, 1995).

Briefly, ligand binding was performed in 0.4 ml of 50 mM HEPES, pH 7.4, containing 3 mM MnCl₂, 0.02% BSA, 0.5 nM [³H]-Substance P (30-56 Ci/mmol Amersham), a final membrane protein concentration of 30-50 μg/ml, and the test compounds. The incubation proceeded at room temperature for 40 min and was stopped by filtration. Non-specific binding was determined using excess of substance P (1 μM) and represents about 6-10% of the total binding. IC₅₀ values of each compound were determined by an 8-point 10×-dilution inhibition curve. pKᵢ values were calculated using the K_{D} of [³H]-Substance P determined in a separate experiment.

The compound of the invention is further characterised in a functional assay for the determination of their effect to inhibit the intracellular calcium increase induced by SP in Human-NK₁-CHO cells using FLIPR technology. Briefly, after 30 minutes incubation with the cytoplasmic calcium indicator Fluo-4 AM (2 μM), cells were washed and incubated in the absence or presence of three or more different concentrations of the antagonist for 60 minutes, at 37° C. in Hank's balanced salts with 20 mM Hepes, and then non-cumulative concentration-response curves of SP (2 μM-300 nM) were performed. The potency of the antagonist (pK_{B} value) was calculated from Schild's analysis.

The action of the compounds of the invention at the NK₁ receptor may be determined by using conventional animal models. Thus the ability to bind at the NK₁ receptor was determined using the gerbil foot tapping model as described by Rupniak & Williams, Eur. J. of Pharmacol., 1994.

The compound of the invention is useful in the treatment of CNS disorders and psychotic disorders, in particular in the treatment or prevention of depressive states and/or in the treatment of anxiety as defined in, but not restricted to, Diagnostic Statistical of Mental Disorder (DSM) IV edition edit by American Psychiatric Association and International Classification Diseases 10th revision (ICD10).

Thus, for example, depressive states depression includes depressive mood episodes, depressive disorders, bipolar disorders, other mood, psychotic and adjustment disorders, premenstrual and dysphroic disorder(PMDD). Thus, for example, depressive mood episodes include major depressive episodes and mixed episodes. Depressive disorders include Major Depressive Disorder (MDD), single or recurrent episodes (with or without psychotic features, catatonic features, melancholic features, atypical features, anxious depression, or postpartum onset), dysthymic disorder (with early or late onset and with or without atypical features) and depressive disorder not otherwise specified. Bipolar disorders include bipolar I and II disorders, cyclothymic disorder and bipolar disorder not otherwise specified. Other mood, psychotic and adjustment disorders include neurotic depression; mood disorders due to general medical conditions including, but not limited to, myocardial infarction, diabetes, miscarriage, abortion, dementia of the Alzheimer's type (with early or late onset) with depressed mood, vascular dementia with depressed mood; substance-induced mood disorders including, but not limited to, depression induced by alcohol, amphetamines, cocaine, hallucinogens, inhalants, opioids, phencyclidines, sedatives, hypnotics, anxiolytics and other substances; schizoaffective disorder of the depressed type; adjustment disorder with depressed mood; adjustment disorder with mixed anxiety and depressed mood.

The term anxiety includes panic attacks, agoraphobia, anxiety disorders, adjustment disorders and separation anxiety disorder and premenstrual dysphroic disorder(PMDD). Thus, for example, anxiety disorders include panic disorder with or without agoraphobia, agoraphobia without a history of panic disorder, specific phobia, social phobia (social anxiety disorder), obsessive-compulsive disorder, acute and post-traumatic stress disorders, generalised anxiety disorders, anxiety disorder due to a general medical condition, substance-induced anxiety disorder, anxiety disorder not otherwise specified and mixed anxiety-depression disorders. Adjustment disorders include adjustment disorder with anxiety and adjustment disorder with mixed anxiety and depressed mood. The compound of the invention is useful as analgesic. In particular, it is useful in the treatment of traumatic pain such as postoperative pain; traumatic avulsion pain such as brachial plexus; chronic pain such as arthritic pain such as occurring in osteo-, rheumatoid or psoriatic arthritis; neuropathic pain such as post-herpetic neuralgia, trigeminal neuralgia, segmental or intercostal neuralgia, fibromyalgia, causalgia, peripheral neuropathy, diabetic neuropathy, chemotherapy-induced neuropathy, AIDS related neuropathy, occipital neuralgia, geniculate neuralgia, glossopharyngeal neuralgia, reflex sympathetic dystrophy, phantom limb pain; various forms of headache such as migraine, acute or chronic tension headache, temporomandibular pain, maxillary sinus pain, cluster headache; odontalgia; cancer pain; pain of visceral origin; gastrointestinal pain; nerve entrapment pain; sport's injury pain; dysmennorrhoea; menstrual pain; meningitis; arachnoiditis; musculoskeletal pain; low back pain e.g. spinal stenosis; prolapsed disc; sciatica; angina; ankylosing spondyolitis; gout; burns; scar pain; itch and thalamic pain such as post stroke thalamic pain.

The compound of the invention is also useful in the treatment of sleep disorders or sleep disturbances including dysomnia, primary insomnia, sleep apnea, narcolepsy, and circadian ritmic disorders or in the treatment of sleep disorders and/or sleep disturbances related or due to other disorders.

The compound of the invention is also useful in the treatment or prevention of the cognitive disorders. Cognitive disorders include dementia, amnestic disorders and cognitive disorders not otherwise specified.

Furthermore, the compound of the invention is also useful as memory and/or cognition enhancers in healthy humans with no cognitive and/or memory deficit.

The compound of the invention is also useful in the treatment of tolerance to and dependence on a number of substances. For example, they are useful in the treatment of dependence on nicotine, alcohol, caffeine, phencyclidine (phencyclidine like compounds) or in the treatment of tolerance to and dependence on opiates (e.g. cannabis, heroin, morphine) or benzodiazepines; in the treatment of addiction to cocaine, sedative ipnotic, amphetamine or amphetamine-related drugs (e.g. dextroamphetamine, methylamphetamine) or a combination thereof.

The compound of the invention is also useful as an anti-inflammatory agent. In particular, it is useful in the treatment of inflammation in asthma, influenza, chronic bronchitis and rheumatoid arthritis; in the treatment of inflammatory diseases of the gastrointestinal tract such as Crohn's disease, ulcerative colitis, inflammatory bowel disease and non-steroidal anti-inflammatory drug induced damage; inflammatory diseases of the skin such as herpes and eczema; inflammatory diseases of the bladder such as cystitis and urge incontinence, overactive bladder and eye and dental inflammation.

The compound of the invention is also useful in the treatment of allergic disorders, in particular allergic disorders of the skin such as urticaria, and allergic disorders of the airways such as rhinitis.

The compound of the invention is also useful in the treatment or prevention of schizophrenic disorders including paranoid schizophrenia, disorganised schizophrenia, catatonic schizophrenia, undifferentiated schizophrenia, residual schizophrenia.

The compound of the invention is also useful in the treatment of emesis, i.e. nausea, retching and vomiting. Emesis includes acute emesis, delayed emesis and anticipatory emesis. The compound of the invention is useful in the treatment of emesis however induced. For example, emesis may be induced by drugs such as cancer chemotherapeutic agents such as alkylating agents, e.g. cyclophosphamide, carmustine, lomustine and chlorambucil; cytotoxic antibiotics, e.g. dactinomycin, doxorubicin, mitomycin-C and bleomycin; anti-metabolites, e.g. cytarabine, methotrexate and 5-fluorouracil; vinca alkaloids, e.g. etoposide, vinblastine and vincristine; and others such as cisplatin, dacarbazine, procarbazine and hydroxyurea; and combinations thereof; radiation sickness; radiation therapy, e.g. irradiation of the thorax or abdomen, such as in the treatment of cancer; poisons; toxins such as toxins caused by metabolic disorders or by infection, e.g. gastritis, or released during bacterial or viral gastrointestinal infection; pregnancy; vestibular disorders, such as motion sickness, vertigo, dizziness and Meniere's disease; post-operative sickness; gastrointestinal obstruction; reduced gastrointestinal motility; visceral pain, e.g. myocardial infarction or peritonitis; migraine; increased intercranial pressure; decreased intercranial pressure (e.g. altitude sickness); opioid analgesics, such as morphine; and gastro-oesophageal reflux disease (GERD) such as erosive GERD and symptomatic GERD or non erosive GERD, acid indigestion, over-indulgence of food or drink, acid stomach, sour stomach, waterbrash/regurgitation, heartburn, such as episodic heartburn, nocturnal heartburn, and meal-induced heartburn, dyspepsia and functional dyspepsia.

The compound of the invention is also useful in the treatment of gastrointestinal disorders such as irritable bowel syndrome, gastro-oesophageal reflux disease (GERD) such as erosive GERD and symptomatic GERD or non erosive GERD, acid indigestion, over-indulgence of food or drink, acid stomach, sour stomach, waterbrash/regurgitation, heartburn, such as episodic heartburn, nocturnal heartburn, and meal-induced heartburn, dyspepsia and functional dyspepsia (such as ulcer-like dyspepsia, dysmotility-like dyspepsia and unspecified dyspepsia), chronic constipation; skin disorders such as psoriasis, pruritis and sunburn; vasospastic diseases such as angina, vascular headache and Reynaud's disease; cerebral ischeamia such as cerebral vasospasm following subarachnoid haemorrhage; fibrosing and collagen diseases such as scleroderma and eosinophilic fascioliasis; disorders related to immune enhancement or suppression such as systemic lupus erythematosus and rheumatic diseases such as fibrositis; and cough.

The compound of the invention is also useful in premenstrual dysphoric disorder (PMDD), in chronic fatigue syndrome and Multiple sclerosis.

The compound of the invention may be administered in combination with other active substances such as anti-nauseants (including 5HT3 antagonists and dexamethasone), serotonin agonists, selective serotonin reuptake inhibitors (SSRI), noradrenaline re-uptake inhibitors (SNRI), tricyclic antidepressants dopaminergic antidepressants or inhibitors of sodium channels.

Suitable 5HT3 antagonists which may be used in combination with the compound of the invention include for example ondansetron, granisetron and metoclopramide.

Suitable serotonin agonists which may be used in combination with the compound of the invention include sumatriptan, rauwolscine, yohimbine and metoclopramide.

Suitable SSRIs which may be used in combination with the compound of the invention include fluoxetine, citalopram, femoxetine, fluvoxamine, paroxetine, indalpine, sertraline and zimeldine.

Suitable SNRIs which may be used in combination with the compound of the invention include venlafaxine and reboxetine.

Suitable tricyclic antidepressants which may be used in combination with the compound of the invention include imipramine, amitriptiline, chlomipramine and nortriptiline.

Suitable dopaminergic antidepressants which may be used in combination with the compound of the invention include bupropion and amineptine.

Suitable inhibitors of voltage-gated sodium channels which may be used in combination with the compound of the invention include lamotrigine, carbamazepine, and phenytoin.

It will be appreciated that the two or more compounds of the combination or composition may be administered simultaneously (either in the same or different pharmaceutical formulations) or sequentially.

The invention therefore provides the compound of formula (I) or a pharmaceutically acceptable salt or solvate thereof for use in therapy, in particular in human medicine.

There is also provided as a further aspect of the invention the use of the compound of formula (I) or a pharmaceutically acceptable salt or solvate thereof in the preparation of a medicament for use in the treatment of conditions mediated by tachykinins (including substance P and other neurokinins).

There is also provided as a further aspect of the invention the use of the compound of formula (I) or a pharmaceutically acceptable salt or solvate thereof in the treatment of conditions mediated by tachykinins (including substance P and other neurokinins)

In an alternative or further aspect there is provided a method for the treatment of a mammal, including man, in particular in the treatment of conditions mediated by tachykinins, including substance P and other neurokinins and comprising administration of an effective amount of the compound of formula (I) or a pharmaceutically acceptable salt thereof.

It will be appreciated that reference to treatment is intended to include prophylaxis as well as the alleviation of established symptoms.

The compound of formula (I) may be administered as the raw chemical but the active ingredient is preferably presented as a pharmaceutical formulation.

Accordingly, the invention also provides a pharmaceutical composition which comprises at least one compound of formula (I) or a pharmaceutically acceptable salt or solvate thereof formulated for administration by any convenient route. Such compositions are preferably in a form adapted for use in medicine, in particular human medicine, and can conveniently be formulated in a conventional manner using one or more pharmaceutically acceptable carriers or excipients.

Thus, the compound of formula (I) may be formulated for oral, buccal, parenteral, topical (including ophthalmic and nasal), depot or rectal administration or in a form suitable for administration by inhalation or insufflation (either through the mouth or nose).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Preparations for oral administration may be suitably formulated to give controlled release of the active compound.

For buccal administration the composition may take the form of tablets or be formulated in a conventional manner.

The compound of the invention may be formulated for parenteral administration by bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use.

The compound of the invention may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compound of the invention may also be formulated in rectal compositions such as suppositories or retention enemas, e.g. containing conventional suppository bases such as cocoa butter or other glycerides.

The compound of the invention may also be formulated as depot preparations. Such long acting formulations may be administered by implantation (for example subcutaneously or intramuscularly) or by intramuscular injection. Thus, for example, the compound of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compound of the invention may be formulated as solutions for administration via a suitable metered or unitary dose device or alternatively as a powder mix with a suitable carrier for administration using a suitable delivery device.

A proposed dose of the compound of the invention is 1 to about 1000 mg per day. It will be appreciated that it may be necessary to make routine variations to the dosage, depending on the age and condition of the patient and the precise dosage will be ultimately at the discretion of the attendant physician or veterinarian. The dosage will also depend on the route of administration and the particular compound selected.

Thus, for parenteral administration a daily dose will typically be in the range of 1 to about 100 mg, preferably 1 to 80 mg per day. For oral administration a daily dose will typically be within the range 1 to 300 mg, e.g. 1 to 100 mg.

The compound of formula (I), and salts and solvates thereof, may be prepared by the general methods outlined hereinafter.

The compound of formula (I) may be prepared by reductive N-alkylation of the compound of formula (II),

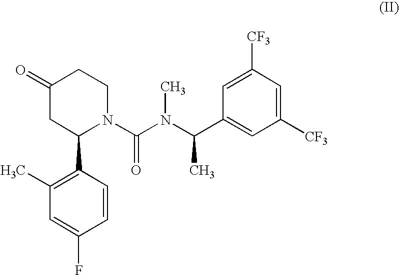

with N-acetylethylenediamine(III) in a solvent such as an alcohol (i.e methanol) and in the presence of a suitable metal reducing agent such as sodium borohydride or sodium triacetoxyborohydride.

Where it is desired to prepare a compound of formula (I) as a salt, for example a pharmaceutically acceptable salt, this may be achieved by reacting the compound of formula (I) in the form of the free base with an appropriate amount of suitable acid and in a suitable solvent such as an alcohol (e.g. ethanol or methanol), an ester (e.g. ethyl acetate) or an ether (e.g. diethyl ether or tetrahydrofuran).

Pharmaceutically acceptable salts may also be prepared from other salts, including other pharmaceutically acceptable salts, of the compound of formula (I) using conventional methods.

The compound of formula (II) is a known compound and its preparation is described in WO 0232867.

In the Intermediates and Examples unless otherwise stated:

Proton Magnetic Resonance (NMR) spectra were recorded on Varian instruments at 300, 400 or 500 MHz, on Bruker instrument at 300 MHz, chemical shifts are reported in ppm (δ) using the residual solvent line as internal standard. Splitting patterns are designed as s, singlet; d, doublet; t, triplet; q, quartet; m, multiplet; b, broad. The NMR spectra were recorded at temperature ranging from 25 to 90° C.; when more than one conformer was detected the chemical shifts for the most abundant one is reported. Mass spectra (MS) were taken on a 4 II triple quadrupole Mass Spectrometer (Micromass UK) or on a Agilent MSD 1100 Mass Spectrometer, operating in ES (+) and ES (−) ionization mode or on a

Agilent LC/MSD 1100 Mass Spectrometer, operating in ES (+) and ES (−) ionization mode coupled with HPLC instrument Agilent 1100 Series [LC/MS - ES (+):analysis performed on a Supelcosil ABZ +Plus (33×4.6 mm, 3 μm) (mobile phase: 100% [water +0.1% HCO₂H] for 1 min, then from 100% [water +0.1% HCO₂H] to 5% [water +0.1% HCO₂H] and 95% [CH₃CN] in 5 min, finally under these conditions for 2 min; T=40° C.; flux=1 mL/min; LC/MS-ES (−):analysis performed on a Supelcosil ABZ+Plus (33×4.6 mm, 3 μm) (mobile phase: 100% [water +0.05% NH₃] for 1 min, then from 100% [water +0.05% NH₃ to 5% [water +0.05% NH₃] and 95% [CH₃CN ] in 5 min, finally under these conditions for 2 min; T=40° C.; flux=1 mL/min]. In the mass spectra only one peak in the molecular ion cluster is reported. Optical rotations were determined at 20° C. with a Jasco DIP360 instrument (I=10 cm, cell volume=1 mL, λ=589 nm). Flash silica gel chromatography was carried out over silica gel 230-400 mesh supplied by Merck AG Darmstadt, Germany or over Varian Mega Be—Si pre-packed cartridges or over pre-packed Biotage silica cartridges.

T.l.c. refers to thin layer chromatography on 0.25 mm silica gel plates (60F-254 Merck) and visualized with UV light. For phase separations performed by using microfiltration devices: phase separation cartridge with polypropylene frit by Whatman or Alltech. SCX means: SCX-cartridges (loading 0.75mmol\g) by Varian.

Solutions were dried over anhydrous sodium sulphate.

Methylene chloride was redistilled over calcium hydride and tetrahydrofuran was redistilled over sodium.

The following abbreviations are used in the text: AcOEt=ethyl acetate, EtOH=ethanol, MeOH=methanol, THF=tetrahydrofuran, NaBH4=sodium borohydride

### EXAMPLE 1

### (2R,4S)-4-{[2-(acetylamino)ethyl]amino}-N-{(1R)-1-(3,5-bis(trifluoromethyl)phenyl)ethyl}-2-(4-fluoro-2-methylphenyl)-N-methyl-1-piperidinecarboxamide

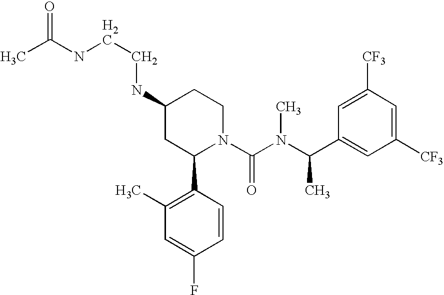

To a solution of 2-(R)-(4-Fluoro-2-methyl-phenyl)-4-oxo-piperidine-1-carboxylic acid, [1-(R)-3,5-bis-trifluoromethyl-phenyl)-ethyl]-methylamide (15g) in 100 ml of EtOH, 8.6 ml of N-acetylethylenediamine was added and the result solution was stirred at room temperature for three hours following the formation of the imine using the MS (ES/+): m/z=589 [M+H]+. Then NaBH4 (1.67g) was added and the mixture was stirred at room temperature for 40 min, then it was carefully quenched with 100 ml of H2O. The reaction was concentrated in vacuo and the aqueous residue was extracted with ethyl acetate. The organic layer was dried and concentrated in vacuo to a residue (mixture of Syn/Anti about 8:2), which was purified by flash chromatography (AcOEt/MeOH 8:2) to give the title compound (4 g) as a white solid.

T.I.c.: AcOEt/MeOH 8:2, Rf=0.25 (detection with ninhydrine).

MS (ES/+): m/z=591 [M+H]+.

NMR-(d₆-DMSO) δ (ppm)

7.97 δ (s, 1H), 7.71 δ (t, 1H), 7.67 δ (s, 2H), 7.13 δ (dd, 1H), 6.88 δ (dd, 1H), 6.75 δ (m, 1H), 5.30 δ (q, 1H), 4.11 δ (d, 1H), 3.33 δ (m, 1H), 3.04 δ (q, 2H), 2.68 δ (m, 1H), 2.68 δ (s, 3H), 2.55 δ (m, 3H), 2.32 δ (s, 3H), 1.90 δ (m, 1H), 1.83 δ (m, 1H), 1.75 δ (s, 3H), 1.80-1.55 δ (bs, 1H), 1.45 δ (d, 3H), 1.36 δ (m, 1H), 1.15 δ (q, 1H).

### PHARMACY EXAMPLE

A. Tablets

| | | |
|---|---|---|
| Active ingredient | 10.0 | mg |
| PVP | 9 | mg |
| Microcrystalline Cellulose | 266 | mg |
| Sodium Starch Glycolate | 12 | mg |
| Magnesium Stearate | 3 | mg |
| Active ingredient | 50 | mg |
| PVP | 9 | mg |
| Microcrystalline Cellulose | 226 | mg |
| Sodium Starch Glycolate | 12 | mg |
| Magnesium Stearate | 3 | mg |

The active ingredient is blended with the other excipients. The blend can be compressed to form tablets using appropriate punches. The tablets can be coated using conventional techniques and coatings.

B. Capsules

| | |
|---|---|
| Active ingredient | 25.0 mg |
| | (1-100 mg) |
| Microcrystalline Cellulose | qs |

The active ingredient is blended with microcrystalline cellulose and then filled into suitable capsules.

C) Injection

| | | |
|---|---|---|
| Active ingredient | 2-20 | mg/mL |
| Buffer solution pH 3.5 (3.0-4.0) suitable for | qs to 10 | mL |
| injection (e.g. citrate buffer in sterile water for | | |
| injection or NaCl 0.9%). | | |

The formulation may be packaged in glass or plastic vials or ampuoles. The formulation may be administered by bolus injection or infusion, e.g. after dilution with D5W or 0.9% NaCl.

The affinity of the compound of the invention for NK₁ receptor was determined using the NK₁ receptor binding affinity method measuring in vitro the compounds' ability to displace [3H]—substance P (SP) from recombinant human NK₁ receptors expressed in Chinese Hamster Ovary (CHO) cell membranes. The affinity value is expressed as negative logarithm of the inhibition constant (Ki) of displacer ligands (pKi).

The pKi value obtained for the compound of the invention as the average of at least two determinations is pKi 10.7

1. A compound of formula (I) 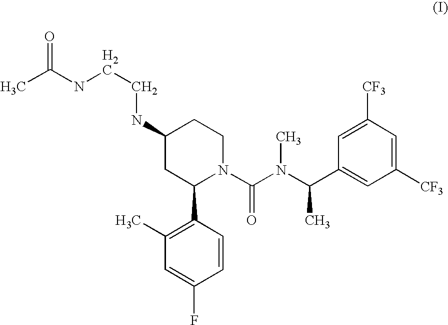
or a pharmaceutically acceptable salt or solvate thereof.

2. The compound as claimed in claim 1 in an isolated form.

3-4. (canceled)

5. A pharmaceutical composition comprising a compound as claimed in claim 1 in admixture with one or more pharmaceutically acceptable carriers or excipients.

6. A method for the treatment of a mammal, including man, in the treatment of depressive states, anxiety, sleep disorders, emesis or overactive bladder, comprising administration of an effective amount of a compound of formula (I) as claimed in claim 1.

7. (canceled)

8. A process for the preparation of the compound as claimed in claim 1, which comprises reductive N-alkylation of the compound of formula (II) with N-acetylethylenediamine(III) 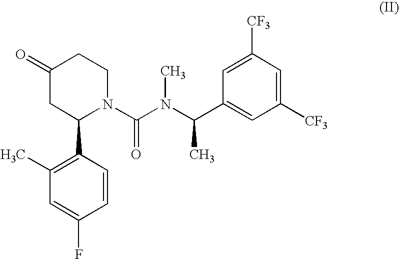
followed where necessary or desired by isolation of the compound as a salt or a solvate thereof.
